(19) United States
(12) Patent Application Publication
(10) Pub. No.: US 2025/0099756 A1
(43) Pub. Date: Mar. 27, 2025

(54) PASSIVE ELECTRICAL NEUROFEEDBACK TREATMENT METHOD FOR MILD TRAUMATIC BRAIN INJURY AND POST-CONCUSSION SYMPTOMS

(71) Applicant: Nexalin Technology, Inc., Houston, TX (US)

(72) Inventor: John Patrick Claude, Redwood City, CA (US)

(21) Appl. No.: 18/890,458

(22) Filed: Sep. 19, 2024

Related U.S. Application Data
(60) Provisional application No. 63/644,314, filed on May 8, 2024.
(60) Provisional application No. 63/584,568, filed on Sep. 22, 2023.

(51) Int. Cl. A61N 1/36

(52) U.S. Cl. CPC A61N 1/36025; A61N 1/36031; A61N 1/36034

(57) ABSTRACT

Electrostimulation systems and methods are contemplated in which a high current level, charge balanced alternating current electrical signal is generated for delivery to a patient's brain for the treatment of mild traumatic brain injury (mTBI) and post-concussion symptoms. By stimulating the brain with a charged balanced stimulation current with a stimulation current envelope defining one or more series of pulses at particular frequencies and durations designed to evoke metabolic response in the neurons, significant improvements in efficacy and reductions in symptoms and pain may be achieved relative to earlier treatment techniques. Further advantages, especially in promoting neural entrainment, may be realized as well via utilizing multiple series of pulses at different frequencies, and via the dynamic adjustment of the stimulation waveform via incorporation of feedback signals in order to maintain charge balance in real-time.

### CROSS-REFERENCE TO RELATED APPLICATIONS

This application relates to and claims the benefit of U.S. Provisional Application No. 63/584,568 filed Sep. 22, 2023, and entitled “MEG Source Imaging Reveals Neuronal Changes in Combat-related Mild Traumatic Brain Injury after Transcranial Electrical Stimulation”, the entire disclosure of which is hereby wholly incorporated by reference, and this application further relates to and claims the benefit of U.S. Provisional Application No. 63/644,314 filed May 8, 2024, and entitled “MEG Source Imaging Reveals Neuronal Changes in Combat-related Mild Traumatic Brain Injury after Transcranial Electrical Stimulation”, the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present disclosure relates generally to the field of electrostimulation. More particularly, the present disclosure relates to improved methods for electrostimulation for treating mild traumatic brain injury and other related conditions.

### 2. Related Art

Electrostimulation devices for applying current to a patient through electrodes located on the head have been developed and used for a variety of purposes in the past, such as for producing analgesic effects, inducing sleep, and reducing or controlling migraine headaches. Often, such treatments are referred to as transcranial electrostimulation (tCES) or cranial electrostimulation (CES). Conventional tCES devices, although employed for a number of different purposes, may have severe drawbacks. For example, many conventional tCES devices utilize a direct current (DC) component in order to break down or lower the resistance of the skin and to allow the treatment current (which may a combination of direct and alternating current) to penetrate to the nervous system.

The presence of a DC component of a treatment current produced by a tCES device generally results in an unpleasant experience for a patient undergoing tCES therapy. In early tCES designs, the presence of the DC current invariably would result in intense pain or burns to the skin of the wearer, requiring the placement of thick conductive padding between the electrodes and the skin of the wearer in order to render the treatment bearable. Even in more recently developed tCES therapies in which the levels of DC current are limited, these limited amounts of DC current still often result in substantial user discomfort. Additionally, even when only an alternating current is applied to the skin, the layers of the skin generally result in a non-linear, complex impedance that invariably rectifies the AC signal and generates a DC component. This DC component depolarizes nociceptors in the skin, causing discomfort in the patient. If the DC-stimulated nociceptors are efferent to a trigeminal nerve branch in the head, the discomfort may be projected into the forehead region.

This patient discomfort resulting from DC rectification presents an upper limit on the amount of power that can be delivered even in an AC-only tCES therapy. Because of this upper limit on power, such conventional therapies are limited in their efficacy.

Transcutaneous electrical nerve stimulation (tENS) is another technique by which electrostimulation devices apply current to patients through electrodes. tENS devices use lower voltage electrical current to provide short-term, non-invasive pain relief by delivering electrical impulses through the skin in order to stimulate nerves in the area in which electrodes are placed. Typically, tENS therapy relies on lower voltage electrical currents compared to transcranial stimulation, which relies on deeper penetration, and as such pain due to the electrical current itself is not typically reported. The exact mechanism of action of TENS therapy is still a matter of debate. One theory is that the transcutaneous delivery of electrical current across the nerves causes overstimulation of the affected nerves, diminishing or eliminating the capability of the affected nerves to perceive pain, and effectively “masking” pain that would otherwise be perceived nerves in the stimulated region. Another theory is that the nerve stimulation caused by the application of tENS therapy stimulates the production of endorphins, which independently act to block pain.

Conventional tENS devices and tENS electrodes, however, have only typically been used to treat pain in regions such as the extremities or the lower back. tENS devices typically carry strong warnings against application in regions such as the head, shoulders, or neck. Recently, advances in tCES system and methods of administering tCES therapy utilizing such systems have allowed for effective treatment of a number of different conditions. Applicant's recently issued U.S. Pat. No. 11,872,397, issued on Jan. 16, 2024, and entitled “Transcranial Alternative Current Dynamic Frequency Stimulation (TACS) system” describes such a system. Applicant's recently issued U.S. Pat. No. 11,944,806, issued on Apr. 2, 2024, and entitled “Transcranial alternating current dynamic frequency stimulation method for anxiety, depression, and insomnia (ADI)”, describes methods of utilizing such systems to treat anxiety, depression, and insomnia. Applicant's U.S. patent application Ser. No. 17/375,555, filed on Jul. 14, 2021, and entitled “Alternating current dynamic frequency stimulation system and method for opioid use disorder (oud) and substance use disorder (sud)”, currently pending, describes methods of utilizing such systems to treat opioid use disorder and substance use disorder. The disclosures of each of these above references are hereby wholly incorporated by reference.

Mild traumatic brain injury (mTBI) is a leading cause of sustained physical, cognitive, emotional, and behavioral deficits in veterans and the general public. However, the underlying pathophysiology is not completely understood, and there are few effective treatments for post-concussive symptoms (PCS) which may result from mTBI. In addition, PCS and post-traumatic stress disorder (PTSD) symptoms overlap considerably. Many studies also report higher rates (nearly double) of comorbid PTSD in individuals with mTBI in both military and civilian settings, compared to those with PTSD without mTBI. The pathophysiology of mTBI is not completely understood, the long-term effects of mTBI are controversial, and there have been few effective treatments for mTBI. Although post-concussion symptoms (PCS) in mTBI resolve by three months after injury in the majority of individuals, about 20% (ranging from 8-33%) of mTBI patients show persistent PCS and long-term cognitive and behavioral impairments. It is unclear why similar mTBI events can lead to dramatic neurobehavioral decompensation with persistent PCS in some patients, but not in others.

Diffuse axonal injury (DAI) commonly induced by sudden acceleration-deceleration or rotational forces is considered to be a major contributor to PCS and cognitive deficits in traumatic brain injury (TBI) patients. Tissue injury is characterized by axonal stretching, inflammation, disruption, and separation of nerve fibers, but axotomy is relative rare in even severe TBI. Abnormalities in neurochemical systems such as cholinergic pathways is another signature of TBI. Measures of functional connectivity (FC) are also of keen interest in mTBI because they are sensitive to disturbances in the communication amongst brain regions. Although disturbances in FC have been reported in many studies of mTBI, there have been significant discrepancies in the findings. Thus, it may be seen that multiple mechanisms may underly disturbances in functional and potentially structural connectivity in mTBI.

Therefore, novel systems and methods for electrostimulation are desirable for treating mTBI and PCS.

### BRIEF SUMMARY

To solve these and other problems, novel methods for treating a patient for mild traumatic brain injury (mTBI) and post-concussion symptoms (PCS) are contemplated in which an electrostimulation system produces a dual symmetric charge balanced alternating current electrical signal for delivery to the frontal cortex region of a patient's brain. By stimulating the frontal cortex region of the brain with a charged balanced AC stimulation current having a stimulation current envelope defining a series of pulses having a particular frequency, with the stimulation current being delivered for a particular duration, together designed to evoke particular metabolic responses in the neurons, significant improvements in efficacy and reductions in patient discomfort may be achieved relative to earlier methods of electrical stimulation, especially those in which a direct current or a resultant rectified direct current component is administered to the patient. Further advantages, especially in promoting neural entrainment, may be realized as well via delivery of the charged-balanced stimulation current such that its envelope defines multiple series of pulses at different frequencies, and via the dynamic alteration of the stimulation current via incorporation of feedback signals in order to maintain charge balance in real-time, in order to maintain charge balance.

Methods for treating a patient for mTBI and PCS are contemplated, with such methods comprising the steps of: (a) generating a carrier waveform, the carrier waveform being an alternating current having a duty cycle ratio and a current amplitude ratio, the first duty cycle ratio and the first current amplitude ratio being selected such that each respective integration of the current amplitude between successive time instances at which the first waveform alternates polarity is substantially equivalent; and generating a stimulation current from the carrier waveform via amplitude modulation the carrier waveform, the extremes of the stimulation current defining a stimulation current envelope, the stimulation current envelope defining a first series of pulses occurring at a first frequency; and (b) applying the stimulation current to the frontal cortex region of the brain of the patient. According to particular refinements of such methods, the first series of pulses may occur at a frequency between 4 Hz and 100 Hz.

The step of generating a stimulation current may, in additional embodiments, occur via amplitude modulating the carrier waveform such that the stimulation current envelope current further defines a second series of pulses occurring at a second frequency. The frequency of the second series of pulses may be selected from a frequency between 4 Hz and 100 Hz.

The above-described methods may also comprise applying the stimulation current to one or more regions of the brain of a patient for a treatment duration, wherein the first series of pulses occur at a predefined frequency that does not substantially vary for the entire treatment duration, and wherein the second series of pulses occur at a second frequency that varies during the treatment duration. According to certain particular exemplary embodiments, the second frequency may vary in accordance with predefined frequency levels corresponding to different portions of the treatment duration. The treatment duration may be, for example, about an hour, with the different portion of the treatment duration being a first portion of the treatment duration, a second portion of the treatment duration, and the third portion of the treatment, each being about 20 minutes.

According to further refinements of the above-described methods, the step of generating the stimulation current may be performed via amplitude modulating the carrier waveform such that the stimulation current envelope defines a plurality of series of pulses, each respective one of the plurality of series of pulses occurring at a respective frequency. Such frequencies may be selected from between 4 Hz and 100 Hz. Further, it is contemplated that the carrier waveform may have a frequency of about 100 KHz, may be a rectangular wave, or both.

In further refinements of the above-described methods, additional steps may be included such as: measuring the stimulation current at the patient, determining of an electrode contact impedance therefrom, and based upon the determined electrode contact impedance, adjusting one or more of: the waveform output from the waveform generator, the stimulation current output from the stimulation current generator, or combinations thereof.

### BRIEF DESCRIPTION OF THE DRAWINGS

FIG. 1 is an illustration showing an embodiment of a high frequency (100 KHz) rectangular alternating current carrier waveform that is charge-balanced, in that the area under the curves (AUC) for each respective integration of current amplitude between successive time instances at which the carrier alternates polarity is equal, with such charge balance resulting from the choice of a particular duty cycle ratio (T2:T1) and a particular current amplitude ratio (Ma (1):Ma (2)) for the carrier waveform;

FIG. 2 is an illustration showing one embodiment of stimulation current comprising the result of amplitude modulating the carrier waveform of FIG. 1 such that the extremes of the stimulation current define a stimulation current envelope, with the stimulation current envelope defining a first series of pulses occurring at a frequency F1 and having a pulse width PW1.

FIG. 3 is an illustration showing another embodiment of a stimulation current comprising the result of amplitude modulating the carrier waveform of FIG. 1 such that the extremes of the stimulation current define a stimulation current envelope, with the stimulation current envelope defining a first series of pulses occurring at a frequency F1 and having a pulse width PW1, and defining a second series of pulses occurring at frequency F2 and having a pulse width PW2;

FIG. 4 is an illustration showing an example of a resultant rectified charge that is entrained at the neurons within the frontal cortex region of a patient's brain, overlaid atop the example of the stimulation current shown in FIG. 2, the resultant rectified charge occurring as a consequence of transcranial application of the illustrated stimulation current to frontal cortex region of a patient's brain.

FIG. 5 is a flowchart showing certain steps of an embodiment of a method for treating a patient for mTBI and PCS;

FIG. 6 is a block diagram showing certain hardware components of an embodiment of a electrostimulation system for treating a patient for mTBI and PCS;

FIG. 7 is a block diagram showing, in greater detail, certain hardware and/or software components of a stimulation circuitry PCB included in one embodiment of a transcranial electrostimulation system for treating a patient for mTBI and PCS;

FIG. 8 is a block diagram showing certain hardware and/or software components of a front panel of an embodiment of an electrostimulation system for treating a patient for mTBI and PCS; and

FIG. 9 is an exemplary image of a front panel user interface of an embodiment of an electrostimulation system for treating a patient for mTBI and PCS.

### DETAILED DESCRIPTION

According to various aspects of the present disclosure, new methods for treatment of mild traumatic brain injury (mTBI) and post-concussion symptoms (PCS) via electrostimulation are contemplated in which a “symmetric” or charge balanced AC signal is delivered to the patient in a manner that permits higher levels of overall power to be transmitted more deeply into the brain without the limitations of the patent discomfort threshold, permitting evocation of nerves in the deep brain structures and enhancing treatment outcome. This increase in power may enhance treatment efficacy and response without any adverse clinical sequelae. By amplitude modulating the carrier waveform to incorporating a blend of multiple frequency patterns of the series of pulses defined by the stimulation current envelope into the treatment, such as a first frequency pattern at a constant frequency for an entire one hour treatment duration, and a second frequency pattern having a varying frequency, the blended frequency pattern of the stimulation current envelope may result in metabolic cleansing and regeneration in damaged neurons, which in particular may be seen to treat both the symptoms and underlying causes of mTBI and PCS.

Turning now to FIG. 1, an exemplary embodiment of a rectangular alternating current (AC) carrier waveform is illustrated. As may be seen, the exemplary rectangular AC carrier waveform has, between each successive alternation of polarity, an area under the curve (AUC), i.e., the integration of the current amplitude between successive time instances at which the waveform alternates polarity. For the waveform to be “symmetric” or “change balanced,” each successive pair of AUC between polarity shifts in the AC waveform must be equal. Via the original rectangular AC carrier waveform being charged balanced in this fashion, it may be seen that the ultimate stimulation current derived from amplitude modulating this waveform will not result in undesired rectification when applied to the patient's skin and thus will not result in the production of a DC component that will cause discomfort or pain in the patient.

The carrier waveform itself may be any type of alternating current waveform. In the exemplary embodiment of FIG. 1, it may be seen that the waveform is generally in the form of a rectangular wave. However, it may be seen that other waveform types may be used as carrier waveforms, such as sinusoidal or triangular waves. It may also be seen that this charge balancing, wherein the AUC of each successive pair of region between alternation in polarity are substantially equivalent, may be achieved in a variety of ways, such that each respective pair of waveforms is not necessarily required to have the same geometry as those preceding it. As shown in the image, the “on” positive amperage portions of the illustrated carrier waveform have a greater magnitude than the “off” negative amplitude portions, but are of a shorter duration (T2), with the duration of the “off” negative amperage portions of the carrier waveform being longer (T1-T2) and with a lesser magnitude. This ratio of the time when the carrier waveform “on” versus “off” is referred to as the duty cycle ratio, which is calculated here, when a rectangular AC waveform is used as the carrier wave, as (T2)/(T1). It may be seen that by controlling the relative magnitudes of the amperages, durations, and possibly even the shape itself of the carrier waveform or portions thereof (especially in non-square waveforms), a carrier waveform may be achieved that is charge balanced. Thus it may be seen that in the case of a dynamic signal, when the duty cycle ratio (T2/T1) of the waveform is changed, so must the current amplitude ratio (mA (1)/mA (2)) also be changed to compensate and maintain the charge balanced nature of the waveform in order to prevent the ultimate stimulation current produced from producing a rectified DC component at the patients' skin when applied to the patient. For example, with a rectangular waveform, the following Table 1 shows various duty cycles and corresponding carrier amplitude ratio pairings that will result in a charge balanced waveform:

TABLE 1
Charged Balanced Duty Cycle Ratio and
Current Amplitude Ratio Pairings

| Duty Cycle Ratio | Current Amplitude Ratio |
|---|---|
| 1:3 | 2:1 |
| 1:4 | 3:1 |
| 1:5 | 4:1 |

In the exemplary embodiment, the carrier waveform is a high-frequency rectangular alternating current, which has a frequency of about 100 KHz. It has generally been found that use high frequency carrier waveform is most beneficial for permitting deep penetration of the stimulation current into targeted regions of the patient's brain. However, in other embodiments, it is contemplated that higher or lower frequencies than 100 KHz may be utilized, without departing from the scope and spirt of the present disclosure. Likewise, it may also be seen that variation in the frequency of the carrier waveform over time or in response to stimuli or other inputs may be utilized in order to enhance the functionality of the transcranial electrostimulation device.

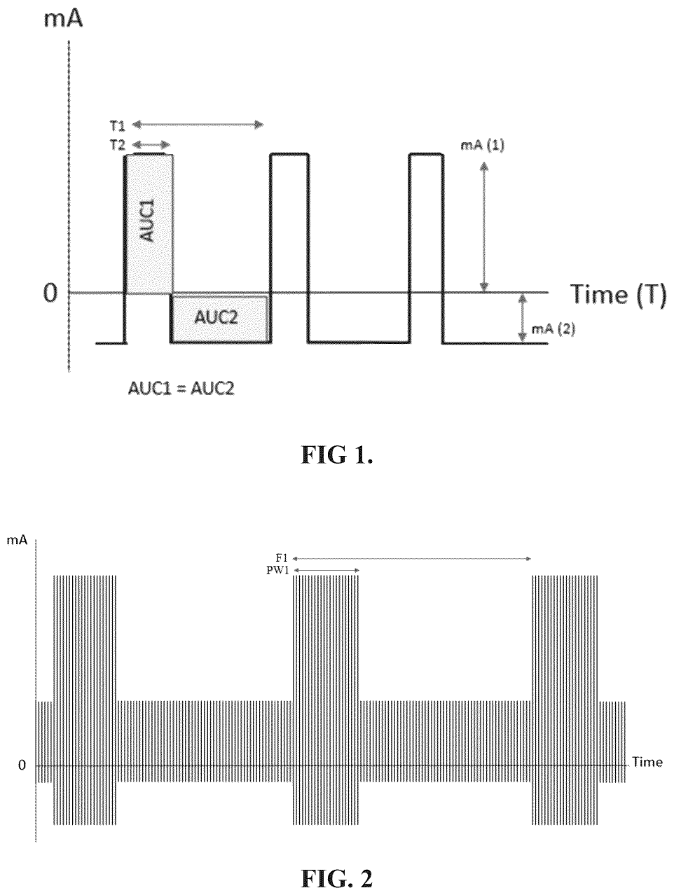

Turning now to FIG. 2, an exemplary embodiment of a stimulation current that has been produced via amplitude modulation of the high frequency carrier waveform of FIG. 1 is illustrated. As may be seen, the extremes of the amplitude modulated high frequency carrier waveform define a stimulation current envelope, which results as a consequence of the particular parameters of the amplitude modulation. The stimulation current envelope may itself be seen to define a first series of pulses occurring at a first frequency F1 and having a pulse width PW2. When applied to the frontal cortex region of the patient's brain, the stimulation current may induce neural entrainment, causing neurons within the patient's brain to be stimulated via polarization of the electrical charge on the outside of the membrane in accordance with the frequency of the first series of pulses. As long as the magnitude and pulse width of the pulses defined by the stimulation current envelope are sufficient to promote neural stimulation and trigger an action potential, and as long as the frequency of the series of pulses are not too high so as to permit the neuron to complete its refractory period prior to excitation via the subsequent pulse, neural entrainment may occur at the neurons that are affected by the stimulation current.

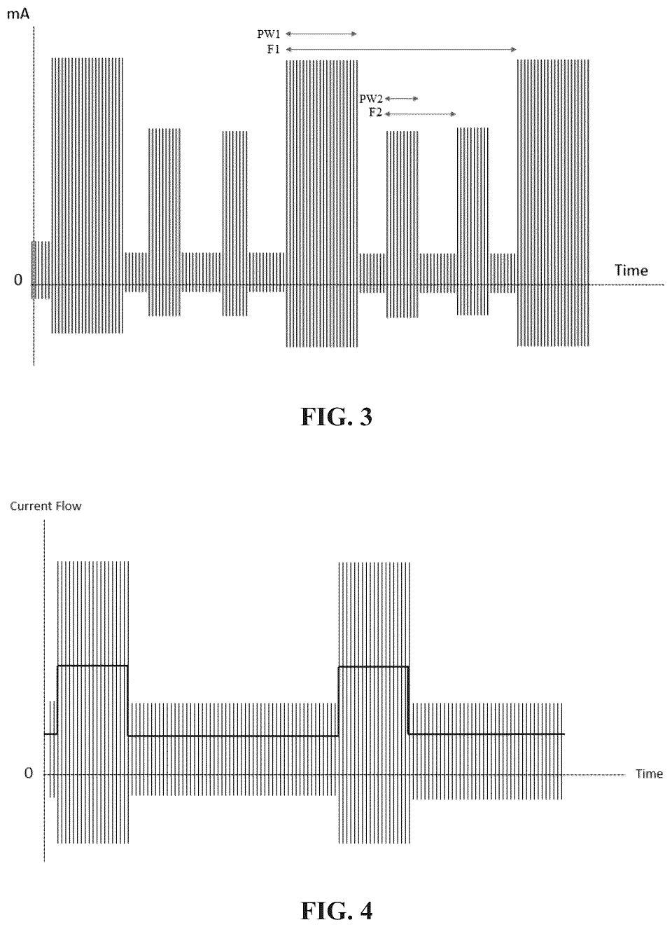

Turning now to FIG. 3, another exemplary embodiment of a stimulation current is illustrated in which the original carrier waveform shown in FIG. 1 has been amplitude modulated such that a first and a second series of pulses are defined by the stimulation current envelope, the first series of pulses having a frequency F1 and a pulse width PW1, and the second series of pulses having a frequency F2 and a pulse width PW2. It may be seen that in this embodiment, the second series of pulses occur at a higher frequency (F2), have a shorter pulse width, and are of a lesser magnitude than the pulses within the first series of pulses. However, it may be seen that in other embodiments of stimulation currents, the pulses of one series of pulses may have higher or lower frequencies, shorter or longer pulse widths, and greater or lesser magnitudes than the pulses of another series of pulses, without departing from the scope and spirit of the presently contemplating disclosure. In this manner, it may be seen that such a stimulation current defining an envelope with multiple series pulses may be created. As a result, by optimizing the parameters of the pulses of each series of pulses, neural entrainment of certain neurons within the patient's brain may be facilitated at Frequencies F1 and/or F1 when the stimulation current is delivered to the patient, with the stimulating current still being charge balanced and not resulting in substantial patient discomfort. By, for example, configuring the stimulation current to have different pulse widths or amplitudes for certain of the series of pulses, it may be seen that certain types or regions of neurons may be targeted by some of the series of pulses for neural entrainment, while other types or regions of neurons may be targeted by others of the series of pulses for neural entrainment.

It may also be seen that other types of schemes for creating a combined stimulation current envelope having other features may be utilized, such as those in which the stimulation current is generated in which the stimulation current envelope defines three or more series of pulses, each series of pulses which may have different parameters in order to facilitate neural entrainment of different types of neurons, or in which the frequencies of the series of pulses defined by the stimulation current envelope are adjustable or configured to adjust according to the receipt of or other feedback, stimuli, or other inputs at the transcranial electrostimulation device.

According to certain exemplary embodiments, in particular it has been discovered that by administering a charge balanced stimulation current which contains a blend of different frequency patterns, neuronal responses within a patient's brain may be evoked which may tend to result in metabolic cleansing and regeneration in damaged neurons. Notably, it is contemplated that administration of a charged balanced stimulation current having a stimulation current envelope that defines a first series of pulses occurring at certain frequencies between 4 Hz and 100 Hz, when delivered to the patient, may tend to evoke a metabolic cleansing response. The particular frequency chosen may differ depending on the particular characteristics of, among other things, the neurons targeted for treatment, the region of the neurons within the patient's nervous system, the particulars of the underlying clinical conditions of the patient, and possible the individual characteristics and needs of the patient. For example, it has been found that in some circumstances, the frequency may be delivered at 4 Hz to achieve a beneficial result. It has also been discovered that a stimulation current having an envelope which defines a series of pulses occurring at a 40 Hz frequency, when delivered to the patient, may tend to promote beneficial results, such as a neuronal regenerative response. Thus, it is contemplated that a stimulation current having a stimulation current envelope that defines both a 4 Hz first series of pulses and a 40 Hz second series of pulses may be delivered to a patient in order to achieve both of these results simultaneously. Further, it is contemplated that by varying the frequency least one of the two series of pulses over time during the administration of a treatment regimen, a synergistic beneficial effect may be realized as a result of the different neural entrainment outcomes resulting from the particular choices used. For example, in one particular embodiment, the stimulation current may have a stimulation current envelope defining a first series of pulses occurring at a constant 40 Hz frequency for the entire duration of the treatment, with the stimulation current envelope also defining a second series of pulses occurring at a variable frequency, the variable frequency being 4 Hz for a first portion of the treatment, 40 Hz for a second portion of the treatment, and 77.5 Hz for a third portion of the treatment. It is further contemplated that for a treatment with a duration of an hour, each of the first, second, and third portions of treatment may be roughly equal, i.e., be 20 minutes in length. As such, the electrostimulation device may be configured to output a stimulation current according to these parameters. It may also be seen that via the delivery of a stimulation current having different frequency and amplitude patterns characteristics of its combined stimulation current envelope, multiple different neural regions may be configured to be stimulated in various ways across a single course of treatment, according to the effects desired to be achieved via such stimulation treatment regimens.

Turning now to FIG. 4, an example of a resultant rectified charge that is entrained at the neurons within the frontal cortex region of a patient's brain as a result of delivery of an exemplary stimulation current to the frontal cortex region of the patient's brain is shown overlaid atop that exemplary stimulation current. It may be seen that this resultant rectified charge may occur as a consequence of application of the illustrated stimulation current to frontal cortex region of a patient's brain, which causes this rectified charge to accrue at the neurons. This rectified charge accrual results in polarization of the electrical charge on the outside of the neural membrane, in accordance with the frequency of the first series of pulses defined by the envelope of the stimulation current. As long as the magnitude and pulse width of the pulses defined by the stimulation current envelope are sufficient to cause sufficient accrual of electrical charge at a neuron to elevate the resting potential of the neuron to the threshold of excitation, an action potential of the neuron will be triggered. As may be seen, a higher magnitude pulse of a lesser pulse width may be sufficient to cause enough charge to accrue, or a lower magnitude pule of a greater pulse width may be sufficient, so long as the sufficient voltage is achieved at the membrane of the neuron as a result of delivery of the stimulation current. Further, it may be seen that so long as the frequency of the series of pulses are not too high (i.e., longer than the neuronal refractory period) each pulse will separate trigger another action potential within the neuron in order to cause natural entrainment to the frequency of the first series pulses. It may further be seen, however, that configurations of the different parameters of stimulation currents may result in some pulses being received at some neurons prior to the recovery of the neuronal refractory period resulting from triggering of the action potential by an earlier pulse. Such schemes may be utilized in order to, for example, target entrainment of certain types or localities of neurons according to a first frequency, and to target entrainment of another type or locality of neurons according to a second frequency.

It may further be seen that the electrostimulation current as presently contemplated may be delivered to the frontal cortex of the patient via different methods, or combinations of methods. For example, transcranial methods whereby the electrostimulation current is delivered to the frontal cortex via conduction between electrodes through soft tissue and skull, where a portion of the current penetrates the scalp and is conducted through the brain. However, it may be seen that delivery of the electrostimulation current to the frontal cortex may be achieved in other ways, such as via transcutaneous delivery of the stimulation current between the electrodes, whereby one or more nerves which are efferent to the frontal cortex, or regions thereof, are affected by the stimulation current, thereby resulting in a propagation of the pulses of the stimulation current by the targeted nerves to the frontal cortex, and thus entrainment at the efferent destination of the targeted nerves. In this manner, it may be seen that transcranial and transcutaneous current delivery may be alternate methods to achieve the result in the entrainment of neurons at the frontal cortex or locations within, and further, that such methods may be used in combination with each other to potentially yield further beneficial effects.

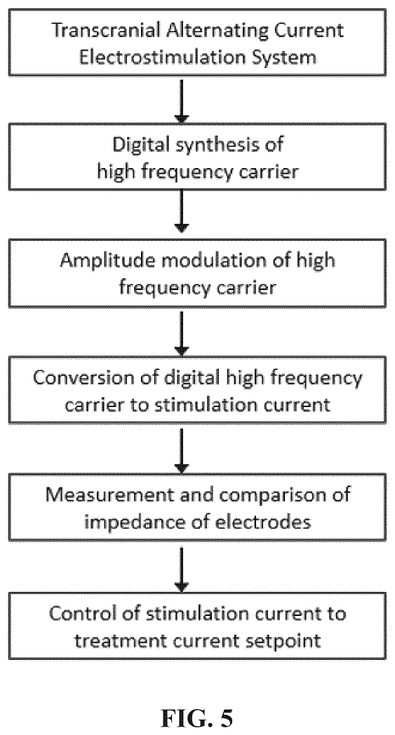

Turning now to FIG. 5, a flowchart showing certain steps of an embodiment of a method for treating a patient for mTBI and PCS via the dynamic delivering a charge balanced alternating current electrical signal to the frontal cortex region of a patient's brain is shown. In particular, it is contemplated that a tCES system may first digitally synthesize one or more high frequency rectangular AC carrier waveforms, which may or may not be similar to the waveform illustrated in FIG. 1. The tCES system may then amplitude modulate the high frequency carrier waveform, as described in detail above, according to the particular parameters ultimately desired in the stimulation current, ultimately producing a stimulation current, which will then be conveyed to the patient. It is further contemplated that in certain embodiments, a measurement of electrode contact impedance may be taken at the patient at the point of delivery of the stimulation current via one or more reference electrodes. In these embodiments, the stimulation current may then be controlled (such as via alternation of the parameters of the high frequency carrier waveform, or by alteration of the various factors of the amplitude modulation) in order to better optimize the performance of the stimulation current, to confirm electrode contact quality, and to prevent any current imbalances that may result in unequal stimulation or inadvertent generation of DC components that may result in discomfort to the patient.

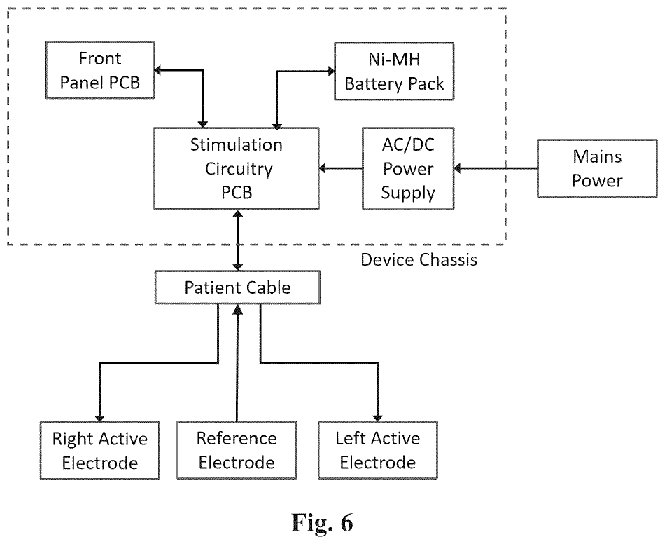

Turning now to FIG. 6, a block diagram of an exemplary Transcranial Alternating Current Dynamic Frequency Stimulation (tACS) system which may be utilized to perform the herein described methods is illustrated. As may be seen, one exemplary embodiment of a tACS system may comprise a device chassis containing an AC/DC power supply, a stimulation circuitry printed circuit board (PCB), a front panel PCB, and a battery pack, configured for use with an external mains power source that feeds into the AC/DC power supply. Also included is a patient cable for conveying the stimulation current to the patient may be attached to the stimulation circuitry PCB, with the patient cable having a right active electrode, a left active electrode, and a reference electrode. While this specific block diagram shows one exemplary version of a tACS system, it is certainly not the only configuration in which the systems and methods herein described may be achieved, and indeed, these descriptions of the actual physical architecture of a tACS system are to be understood as being purely for exemplary purposes in order to enable the reader to more fully understand the nature of the herein described methods, and are not to be interpreted as representing or imposing any limitations of the subject matter described herein. For example, but without limitation, it may not be necessary for some or all components to be contained within a physical device chassis, or for many of these components to be present in the exact form described or at all.

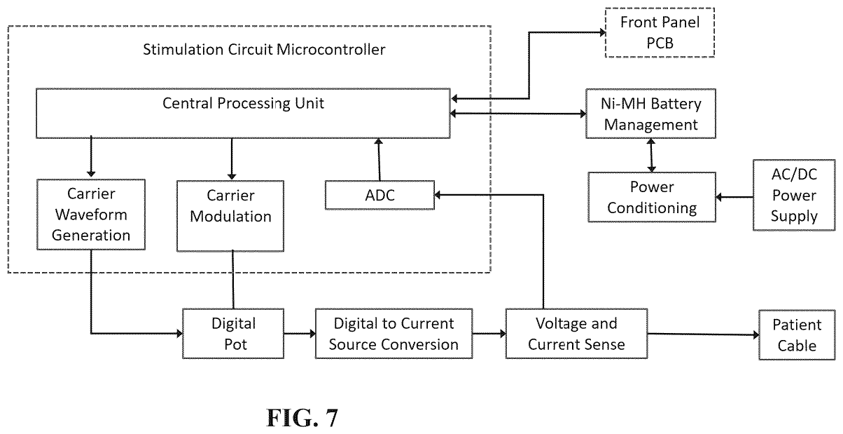

The stimulation circuitry PCB may be for controlling the functionality of the tACS related to the generation and control of the stimulation current, including the synthesis of a high frequency carrier waveform. In this respect, it is to be understood as including as subsidiary components (which may be hardware or software components, or combinations thereof) both the waveform generator and the stimulation current generator. The stimulation circuitry PCB will be more fully described in relation to the foregoing discussion of FIG. 7.

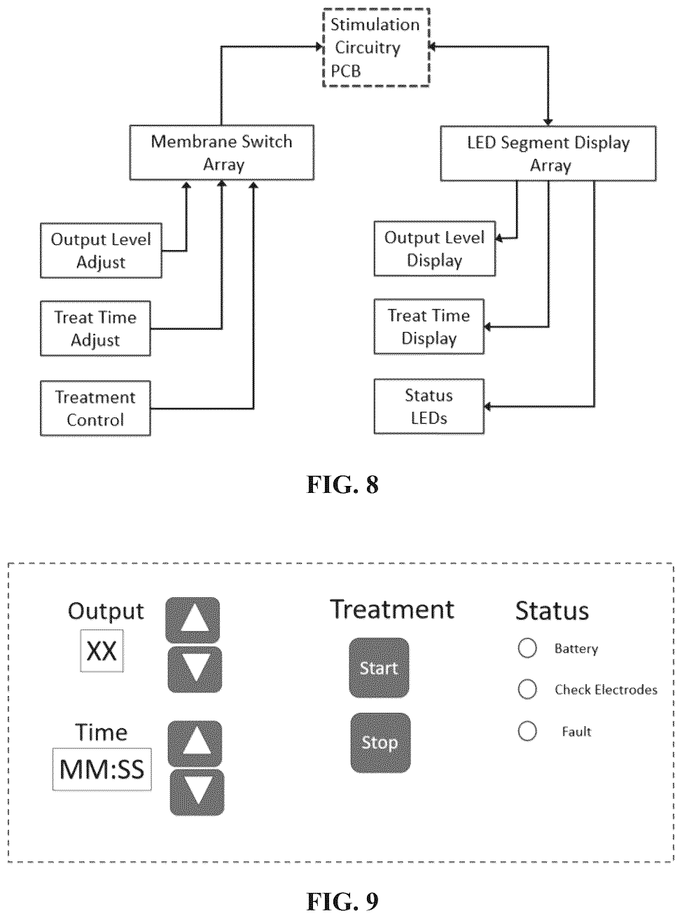

The front panel PCB may be for supporting the user interface for the tACS system, and may include, for example, means for user input and for display of information to the user. The front panel PCB will be more fully described in relation to the foregoing discussion of FIG. 8.

The patient cable may be for conveying the stimulation current produced at the tACS to the patient. The patient cable may include or be connected to two or more active electrodes for delivering the stimulation current to the patient, and may further include or be connected to one or more reference electrodes for determining stimulation output voltage and returning measurements which will be used to determine electrode impedance. The active electrodes may comprise a pliable substrate with an electrically conductive adhesive. In an exemplary embodiment, the active electrodes may be applied to the left and right mastoid region of the patient, with the reference electrode applied to the patient's forehead. However, it may be seen that in other configuration which may be optimized for other types of stimulation, the location, positioning, quantity, etc. of the active electrodes and the reference electrode(s) may be different.

The power supply, which in the exemplary embodiment may be optional and which may be a medical grade AC/DC power supply, may be any power supply or other which may be used to receive mains power and to permit that mains power to be conveyed the remainder of the system and utilized to ultimately produce a stimulation current. Likewise, the battery pack, which again may be an optional component, and which in the exemplary embodiment is a Ni-MH battery pack that also includes a battery management system, may serve to provide uninterrupted power during mains power failure, and which may serve to prevent artifact generation (spikes, jitters, etc.) that may occur during failure or intermittent losses or reduction in mains power delivery, as such artifacts may be included within the stimulation current which may result in inadvertent rectification of the stimulation by the skin and production of a DC current component, leading to patient discomfort. However, it may be seen that the presence or absence of these components are not of critical importance to the systems or methods herein disclosed, and that, such systems or methods may be performed without a battery pack or a power supply, so long as the mains power or other source of current used to produce the stimulation current is sufficient to enable performance of the herein discussed methods.

Turning now to FIG. 7, a block diagram showing, in greater detail, certain hardware and/or software components of a stimulation circuitry PCB according to one embodiment of a transcranial electrostimulation system which may be utilized to perform the herein described methods for treating a patient for mild traumatic brain injury (mTBI) and post-concussion symptoms. As may be seen, the stimulation circuitry PCB may, in the exemplary embodiment shown, include a stimulation circuit microcontroller comprising a central processing unit (CPU), a waveform generator module, a waveform modulation module, and an analog to digital converter (ADC) module, with the stimulation circuitry PBC also including a digital to current source converter module, a voltage and current sense module, a digital potentiometer (pot), a Ni-MH battery management module, a power conditioning module, and inputs/outputs to the front panel PCB and to the patient cable. While this specific block diagram shows one exemplary version of the stimulation circuitry of a tACS system, it is certainly not the only configuration in which the systems and methods herein described may be achieved, and indeed, these descriptions of the physical and/or digital architecture of the stimulation circuitry of a tACS system are to be understood as being purely for exemplary purposes in order to enable the reader to more fully understand the nature of the herein described methods, and are not to be interpreted as representing or imposing any limitations of the subject matter described herein. It is also to be understood that the respective modules described herein may be implemented in hardware, in software, or in combinations of hardware and software, including as subsidiary components of one another or integrated together.

The CPU may provide software control of all hardware functions in the tACS system. The CPU may also receive inputs from the ADC module and perform calculations based upon those inputs in order to control the functionality of the tACS system and its subordinate components in real time.

The carrier waveform generator module may be controlled by the CPU and may generate a carrier waveform according to the specific parameters desired, which may include a duty cycle and current amplitude ratio. The carrier waveform may then be then amplitude modulated with a carrier waveform via a digital potentiometer controlled by a waveform modulation model (also potentially controlled by the CPU) to perform the herein described steps in order to produce a digital representation of the herein described stimulation current. According to a preferred embodiment, the carrier waveform and thus the resulting stimulation current has a frequency of about 100 KHz.

Following amplitude modulation of the carrier waveform, a digital to current source converter, i.e., the stimulation current generator, may be used to ultimately generate, from a digital representation of the amplitude modulated carrier waveform, the actual stimulation current for subsequent delivery to the patient. According to a preferred embodiment, the stimulation current is about 15 mA. However, it may be seen that the stimulation current flow may also be at different rates.

The ADC module may be configured to receive analog information from a voltage and current sense module and to convert that analog information to digital information for use by the CPU in order to permit real-time adjustment of the stimulation current. Such analog information may be, according to certain contemplated embodiments, information received from an active electrode or a reference electrode, which may concern quality of electrode contact, electrical impedance, etc. Such information may be used to provide feedback to the CPU and to permit dynamic adjudgments to be made in real time to the stimulation current, such as via adjustment of the underlying waveform, the modulation signal(s), or directly at the stimulation current itself.

In the exemplary embodiment, a power conditioning module may also be included within or in relation to the stimulation circuitry PCB for regulating the power supply to voltage supply rails for the operation of the microcontroller and the stimulation output circuitry.

Turning now to FIG. 8, a block diagram is illustrated that shows certain hardware and/or software components of a front panel of an exemplary embodiment of a transcranial electrostimulation system which may be utilized to perform the herein described methods of treating a patient for mild traumatic brain injury (mTBI) and post-concussion symptoms. In this exemplary embodiment, the front panel may be seen to include a membrane array switch and a LED segment display array. The membrane array switch may be utilized by the user of the TECS system in order to manually input adjustments to the parameters of the stimulation current, such as output level, treatment time, or treatment controls. The LED segment display array may be viewed by the user to visually confirm these parameters and the overall status of the device. It is to be understood that a this description of a front panel is purely illustrative in nature and is specific to one exemplary embodiment of a TECS system, and that the presence, absence, or specific configuration of any front panel, or any panel located anywhere on any such device, or the controls or displays contained therein, are purely illustrative of merely one particular embodiment, and these descriptions are certainly not meant to impose any limitations on the inventive aspects of the herein described systems and methods.

Turning now to FIG. 9, an exemplary image of a front panel user interface of an embodiment of a transcranial electrostimulation system for performing the herein described methods of treating a patient for mild traumatic brain injury (mTBI) and post-concussion symptoms is shown. It may be seen that the front panel user interface may, according to the particular embodiment illustrated, include controls from adjusting an output, which may be a current level setpoint (i.e., in milliamperes) or even an alphanumeric signifier that relates to a treatment type, such as the output of a stimulation current according to one of the many aforementioned variations in frequency or multiple frequencies, or an entire set of predefined treatment parameters encompassed within a treatment modality. A treatment time may also be adjusted, as well as a manual start/stop button for beginning or ending the treatment. The front panel user interface may also contain, without limitation, one or more status LEDs for indicating a status condition, such as a battery status (i.e., fully charged, low charge, no charge remaining, etc.), a check electrode status (i.e., no or poor contact of one or more electrodes), or a general fault status which may indicate other conditions not encompassed by other status indicators. However, it is to be understood that a this description of a front panel user interface is purely illustrative in nature and is specific to one exemplary embodiment of a TECS system, and that the presence, absence, or specific configuration of any front panel user interface, or the controls or displays contained thereon or therein, are purely illustrative of merely one particular embodiment, and these descriptions are certainly not meant to impose any limitations on the inventive aspects of the herein described methods.

### Clinical Trials:

Subjects were separated into three groups. Two groups contained OEF/OIF/OND veterans (age 18-60) diagnosed with chronic mTBI with persistent PCS, the majority due to blast exposure, who currently have persistent PCS at least 6 months post-injury. Participants were matched in those two groups on age, gender, education, combat exposure, symptom chronicity, and socioeconomics. Group 1 (mTBI treatment group) contained mTBI veterans. Group 2 (mTBI sham group) contained mtBI veterans assigned to a sham treatment. Group 3 (control group) consisted of age-, gender-, education-, combat exposure-, and socioeconomically-matched veterans.

Exclusion criteria included: 1) a history of other neurological, developmental, or psychiatric disorders (based on the DSM-5 (MINI-7) structured interview), e.g. brain tumor, stroke, epilepsy, Alzheimer's disease, schizophrenia, bipolar disorder, ADHD, or other chronic neurovascular diseases such as hypertension or diabetes; 2) substance or alcohol use disorders according to DSM-5 criteria within the six months prior to the study; 3) history of metabolic or other diseases known to affect the central nervous system; 4) metal objects (e.g. shrapnel or metal fragments) that fail MRI screening, or extensive metal objects in the head, neck, or face area that cause non-removable artifacts in MEG data; 5) subjects reporting a “2” or “3” on item 9 (suicidal thoughts or wishes) of the Beck Depression Inventory (BDI-II) to evaluate levels of depressive symptoms and suicidal ideation.

The total treatment contained 12 sessions at ˜3 sessions/week. In each session, transcranial electrostimulation (tCES) pulses were at 15 mA current, delivered through three electrodes placed at the forehead and left-right mastoids, with three repetition frequencies at 4 Hz, 40 Hz, and 77.5 Hz, at ˜20 minutes for each frequency. This current level is undetectable by the participants. Rs-MEG was conducted at both pre-treatment baseline and post-treatment follow-up exams. MEG is a non-invasive functional imaging technique that directly measures the magnetic signal due to neuronal activation in grey matter with high temporal resolution (<1 ms) and special localization accuracy (2-3 mm at the cortical level). Twenty-four veterans with combat-related mTBI completed the tCES (N=15) or sham (N=9) treatments with both pre—and post-treatment rs-MEG exams. Ten participants completed the 12-session active tCES (N=7) or sham (N=3) treatments but without both pre- and post-treatment rs-MEG, and nine did not finish the active TCES (N=5) or sham (N=4) treatment. The delta (1-4 Hz) and gamma (30-80 Hz) frequency bands were focused on, and a repeated measure ANOVA tested for the treatment effect in rs-MEG data. PCS were assessed using the Rivermead Post-Concussion Symptoms Questionnaire (RPQ), Neurobehavior Symptom Inventory (NSI) and McGill Pain Questionnaire (MPQ).

None of the veterans reported adverse effects, with one participant noticing some discomfort during impedance checking due to poor electrode-skin contact, which was quickly corrected. Compared with the sham group, veterans in the active treatment group showed significant (corrected p<0.01) reduction in: 1) delta-band activity mainly from the frontal pole and inferior frontal gyri, suggesting improvement in deafferentation; and 2) abnormal gamma-band activity mainly from the frontal pole, orbital frontal cortex, and posterior parietal regions, suggesting improvement in GABA-ergic inhibitory interneuron functions. The active treatment group showed reduced RPQ and NSI symptoms (total score) relative to the sham group, but not reaching statistical significance, potentially due to the limited sample size and failure of 10 subjects to fully complete treatment. In contrast, the MPQ score in the treatment group showed significant reduction in pain (pre-vs. post-: p=0.00031). The amount of MPQ reduction in in the treatment group was significantly more pronounced than the sham group (p=0.0027).

These findings demonstrate that MEG is a sensitive and objective functional imaging technique for assessing neuronal changes due to tCES treatment in combat-related mTBI. The reduction of hyperactivity of delta- and gamma-band activities in mTBI suggest that tCES treatment can reduce deafferentation and GABA-ergic inhibitory interneuron dysfunctions in chronic mTBI. The treatment group not only showed a significant reduction in pain, as measured by MPQ, but the reduction in pain was more significant than that in the sham group. A follow-up study with a larger sample size may be required to validate initial results.

### DISCUSSION

Evidence suggests that the waveform that provides tCES to the brain delivered at a frequency of 4 Hz, 40 Hz, and 77.5 Hz at 0 to 15 mA peak current results in improved clinical outcomes in terms of anxiety and pain. The specific mechanisms of action are not yet known, but available evidence suggests that this waveform alters the function of the hypothalamus and related structures. In particular, tCES may lead to increases in levels of enkephalins and beta-endorphins in the brain and in cerebrospinal fluid (CFS). Other data suggests that tCES can alter endogenous levels of both substance P and serotonin. Repeat tCES treatments over time may serve to stimulate long-term neurochemical changes.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the exemplary embodiments.

It is also to be understood that the terminology employed in the Summary of the Invention and detailed description section of this application is for the purpose of describing particular embodiments. Unless the context clearly demonstrates otherwise, it is not intended to be limiting. In this specification and the appended claims, the singular forms of articles shall include plural references unless the context clearly dictates otherwise, and further, it is contemplated that any use of “and” or “or” statements shall be interpreted as interchangeable with one another. It is also contemplated that any optional feature of the variations described herein may be set forth and claimed independently or in combination with any one or more of the features described herein.

What is claimed is:

1. A method for treating a patient for mild traumatic brain injury (mTBI) and post-concussion symptoms (PCS), the method comprising:
generating a carrier waveform, the carrier waveform being an alternating current having a duty cycle ratio and a current amplitude ratio, the duty cycle ratio and the current amplitude ratio being selected such that each respective integration of the current amplitude between successive time instances at which the carrier waveform alternates polarity is substantially equivalent;
generating a stimulation current from the carrier waveform via amplitude modulating the carrier waveform, the extremes of the stimulation current defining a stimulation current envelope, the stimulation current envelope defining a first series of pulses occurring at a first frequency; and
applying the stimulation current to the brain of the patient.

2. The method of claim 1, wherein the first series of pulses occur at a frequency of about 40 Hz.

3. The method of claim 1, wherein the stimulation current is generated via amplitude modulating the carrier waveform such that the stimulation current envelope current further defines a second series of pulses occurring at a second frequency.

4. The method of claim 3, wherein the second series of pulses occur at a frequency selected from: about 4 Hz, about 40 Hz, about 77.5 Hz.

5. The method of claim 3, wherein the method comprises applying the stimulation current to the brain of the patient for a treatment duration, wherein the first series of pulses occurs at a frequency of about 40 Hz for the entire treatment duration, and wherein the second series of pulses occurs at frequency of about 4 Hz for a first portion of the treatment duration, a frequency of about 40 Hz for a second portion of the treatment duration, and a frequency of about 77.5 Hz for a third portion of the treatment duration.

6. The method of claim 5, wherein the treatment duration is about an hour, and wherein each of the first portion of the treatment duration, the second portion of the treatment duration, and their third portion of the treatment duration are about 20 minutes.

7. The method of claim 1, wherein the stimulation current is generated via amplitude modulating the carrier waveform such that the stimulation current envelope defines a plurality of series of pulses, each respective one of the plurality of series of pulses occurring at a respective frequency.

8. The method of claim 7, wherein each of the plurality of series of pulses of the stimulation current envelopes has a frequency selected from one or more of: about 4 Hz, about 40 Hz, about 77.5 Hz.

9. The method of claim 1, wherein the carrier waveform has a frequency of about 100 KHz.

10. The method of claim 1, wherein the carrier waveform is a rectangular wave.

11. The method of claim 1, further comprising measuring the stimulation current at the patient and determining an electrode contact impedance therefrom, and based upon the determined electrode contact impedance, adjusting one or more parameters of one or more of: the carrier waveform output from the waveform generator, the stimulation current output from the stimulation current generator, or combinations thereof.
